(19) United States
(12) Patent Application Publication
(10) Pub. No.: US 2025/0288982 A1
(43) Pub. Date: Sep. 18, 2025

(54) PREPARATION METHOD OF MULTI-CHANNEL Co@CM CERAMIC CATALYTIC MEMBRANE

(71) Applicant: NANJING TECH UNIVERSITY, Nanjing (CN)

(72) Inventors: Rizhi CHEN, Nanjing (CN); Zhihao GUO, Nanjing (CN); Yan DU, Nanjing (CN); Jiuxuan ZHANG, Nanjing (CN); Hong JIANG, Nanjing (CN); Weihong XING, Nanjing (CN)

(73) Assignee: NANJING TECH UNIVERSITY, Nanjing (CN)

(21) Appl. No.: 19/221,566

(22) Filed: May 29, 2025

Related U.S. Application Data
(63) Continuation of application No. PCT/CN2023/119309, filed on Sep. 18, 2023.

(30) Foreign Application Priority Data
Sep. 4, 2023 (CN) 202311126622.4

(51) Int. Cl. B01J 35/59; B01J 23/75; B01J 35/64; B01J 37/00; B01J 37/08; C07C 209/26

(52) U.S. Cl. CPC B01J 35/59; B01J 23/75; B01J 35/651; B01J 35/657; B01J 37/0018; B01J 37/088; C07C 209/26

(57) ABSTRACT

A preparation method of a novel multi-channel Co@CM ceramic catalytic membrane is provided. The catalytic membrane is prepared as follows: a multi-channel ceramic membrane is adopted as a support, and ZIF-67 is assembled layer by layer on a surface and inside pores of the ceramic membrane and then reduced through one-step pyrolysis to produce the catalytic membrane. The preparation method has the following advantages: Nano-scale Co particles are loaded instead of a precious metal on a ceramic membrane, and the surface of the Co particles is wrapped by carbon and nitrogen, which can effectively inhibit the loss of Co particles during a reaction. The prepared Co@CM ceramic membrane has excellent catalytic activity and stability, solves the problem that the traditional catalysts can hardly be separated from products subsequently, and can be widely used in hydrogenation reaction processes.

### CROSS REFERENCE TO THE RELATED APPLICATIONS

This application is a continuation application of International Application No. PCT/CN2023/119309, filed on Sep. 18, 2023, which is based upon and claims priority to Chinese Patent Application No. 202311126622.4, filed on Sep. 4, 2023, the entire contents of which are incorporated herein by reference.

### TECHNICAL FIELD

The present disclosure belongs to the technical field of ceramic membrane-based catalysis, and relates to a preparation method of a multi-channel Co@CM ceramic catalytic membrane.

### BACKGROUND

Membrane separation and catalytic reactions can be coupled to achieve catalytic membrane reactors. A catalytic membrane serves as a core part of a catalytic membrane reactor. Catalytic membranes are characterized in that active components can be loaded on membrane materials to allow the separation of reactants from products through membrane separation while enabling catalytic reactions. Catalytic membranes have the advantage of allowing the in-situ separation of materials from catalysts, which can simplify the process steps and reduce the energy consumption. The development of catalytic membranes primarily focuses on the surface modification and preparation methods for membranes. The patent (CN110841633B) discloses a preparation method of a surface-modified catalytic membrane. Specifically, a ceramic membrane support is first modified with titanium dioxide, then Pd(hfac)₂ is deposited on a surface of a modified ceramic membrane support through atomic layer deposition (ALD), and the precursor is then reduced into Pd with Formalin (an aqueous solution including 37% of formaldehyde and 15% of methanol) to produce the catalytic membrane. This preparation method enhances the activity and stability of the catalytic membrane to some extent. However, precious metals are easily lost during a reaction process and are costly, making it difficult to achieve the large-scale application. Therefore, in addition to these precious metal catalysts, non-precious metal catalysts such as Co have been employed in catalytic reactions. In recent years, metal-organic frameworks (MOFs) have attracted widespread attention as template materials to prepare high-porosity carbonaceous materials. MOFs are a class of novel ordered nanoporous materials assembled from metal ions and organic ligands. MOFs have characteristics such as large specific surface area, ordered pore structures, and pore adjustability. It has also been reported that MOFs can be directly carbonized (without a carbon source) to prepare MOF-derived metals or metal oxide-carbon composites. The patent (CN113289666A) discloses a preparation method of a catalytic membrane. This preparation method is as follows: ZIF-67 is allowed to grow in situ on a surface of a ceramic membrane through impregnation, and then the calcination is conducted in an argon atmosphere, such that a reductive gas generated from the pyrolysis of an imidazole framework reduces Co²⁺ in ZIF-67 in situ into catalytically-active Co⁰. The carbonization of an organic ligand during the pyrolysis of MOF can effectively prevent the agglomeration of metal nanoparticles. Moreover, pyrrolic nitrogen can be generated during the pyrolysis to effectively enhance the catalytic activity of the catalytic membrane. However, compared to powder catalysts, catalytic membranes have a small load capacity for active components, resulting in low catalytic efficiency per unit volume. Compared with flat sheet membranes and single-channel membranes, multi-channel ceramic membranes have abundant pore structures, and thus can provide increased loading sites for active components per unit volume to achieve the improvement for a load capacity, which can significantly improve the catalytic efficiency of a membrane per unit volume. However, the combination of MOFs with ceramic membranes to fabricate catalytic membranes remains challenging. In the catalytic membranes prepared by the conventional methods, there is often a small amount of an active center component per unit volume and there is weak bonding between an active component and a membrane material. As a result, these catalytic membranes have a low catalytic activity and are prone to deactivation in use, which hinders the large-scale industrial application of catalytic membranes.

### SUMMARY

In view of the problems such as low activity and poor stability of the traditional catalytic membranes, the present disclosure proposes a preparation method of a novel multi-channel Co@CM ceramic catalytic membrane.

In order to achieve the above objective, the present disclosure adopts the following technical solutions:

A preparation method of a novel multi-channel Co@CM ceramic catalytic membrane is provided, including the following specific steps:
step 1, dissolving 2-methylimidazole in methanol, and stirring until a resulting solution is clear and transparent to produce a solution A;
step 2, dissolving cobalt nitrate hexahydrate in methanol, and stirring until a resulting solution is clear and transparent to produce a solution B;
step 3, filling channels of a multi-channel ceramic membrane tube with the solution A; under an action of a peristaltic pump, forcing the solution A in the channels to flow through membrane pores of the multi-channel ceramic membrane tube and then flow out of the multi-channel ceramic membrane tube to allow a forced circulation of the solution A for 1 h, wherein a pipeline is designed such that the solution A can be recycled under the action of the peristaltic pump; after the forced circulation of the solution A is completed, discharging the solution A; replacing the solution A with the solution B, and iterating the above operation; and conducting at least two cycles of alternate forced circulations of the solution A and the solution B;
step 4, after the solution A and the solution B are discharged, forcing methanol to flow through a surface and membrane pores of the multi-channel ceramic membrane tube for washing, and oven-drying the multi-channel ceramic membrane tube to produce a multi-channel ZIF-67@CM ceramic membrane;
step 5, calcining the multi-channel ZIF-67@CM ceramic membrane to produce a multi-channel Co@CM ceramic catalytic membrane; and
step 6, rinsing membrane pores through a forced circulation of an ethanol aqueous solution for a specified period of time to remove active components unstably loaded to produce the multi-channel Co@CM ceramic catalytic membrane with stable performance.

Preferably, in the step 1, a concentration of the 2-methylimidazole in the solution A is 0.32 mol/L to 0.64 mol/L.

Preferably, in the step 2, a concentration of the cobalt nitrate hexahydrate in the solution B is 0.04 mol/L to 0.08 mol/L.

Preferably, in the step 3, during the forced circulation, heat preservation is conducted with a water bath at 25° C. to 45° C.; the solution A is preferentially introduced for alternate circulations of the solutions; a total time of the alternate forced circulation flows of the solution A and the solution B is 4 h to 6 h; and a material for the multi-channel ceramic membrane tube is alumina or zirconia, and the multi-channel ceramic membrane tube includes 7 to 61 channels and has a pore size of 200 nm to 5,000 nm.

Preferably, in the step 4, the washing is conducted for 10 min to 20 min, and the oven-drying is conducted at 50° C. to 70° C. for 12 h to 24 h.

Preferably, in the step 5, a pyrolysis temperature is 450° C. to 770° C., a calcination atmosphere is argon, a heating rate is 2° C./min to 10° C./min, and the pyrolysis temperature is held for 4 h to 6 h.

Preferably, in the step 6, the forced circulation for the rinsing is conducted for 45 min at a flow rate of 2.5 L/h; the rinsing is conducted with the ethanol aqueous solution; and in the ethanol aqueous solution, a volume ratio of deionized water to ethanol is 5:1.

In the present disclosure, the prepared catalyst is used in a reaction for selective hydrogenation of p-nitrophenol to produce p-aminophenol to verify the catalytic performance of the catalyst. During the reaction, the catalytic membrane is fixed in a membrane module. After a temperature of a reaction system is controlled, a mixed solvent of ethanol and deionized water is added, the p-nitrophenol raw material is added and dissolved, and then NaBH₄ is added as a reducing agent to initiate the reaction. During the reaction, a sample can be collected multiple times, and a composition of a product is analyzed by high-performance liquid chromatography (HPLC, Agilent 1200). After the reaction is completed, a liquid in a storage tank is discharged, deionized water is introduced instead and forcibly flows through membrane pores for 10 min to 15 min to allow washing, and a catalytic membrane is then taken out, air-dried or oven-dried, and stored. Alternatively, the fresh raw material is added to continue the reaction.

In the present disclosure, with a catalytic membrane composed of ZIF-67 and a multi-channel ceramic membrane substrate as a research object, a novel Co@CM ceramic membrane is prepared through one-step high-temperature pyrolysis. Since ZIF-67 does not include oxygen atoms, carbon and nitrogen atoms are retained in large quantities during calcination in an inert atmosphere to produce a nitrogen-rich catalytic membrane. The nitrogen-rich catalytic membrane facilitates the electron transfer, can effectively suppress the loss of active components, and can exhibit excellent catalytic performance in a reaction for hydrogenation of p-nitrophenol to produce p-aminophenol. In the present disclosure, a pyrolysis temperature is adjusted to effectively prepare a ZIF-67-derived metal-carbon/nitrogen composite. Studies have revealed that Co²⁺ can be reduced into Co by a reductive gas generated during the calcination of ZIF-67 and thus serves as a catalytic active center. The present disclosure effectively prepares a ZIF-67-derived metal-carbon/nitrogen composite by regulating the concentrations of 2-methylimidazole and cobalt nitrate hexahydrate. Studies have shown that, with the increase of a concentration of the raw material, a load capacity of Co increases significantly. The carbonization of an organic ligand during the pyrolysis of MOF effectively avoids the agglomeration of metals. Pyrrolic nitrogen is gradually generated during the pyrolysis, which facilitates the conversion of the reactant.

Compared with the prior art, the present disclosure has the following advantages and positive effects:

1. The multi-channel ceramic membrane has a large specific surface area and enables increased loading sites per unit volume, which increases the load capacity for active components and remarkably enhances the catalytic efficiency per unit volume.

2. Through the layer-by-layer assembly, ZIF-67 can be orderly dispersed on a surface on and in pores of a ceramic membrane, resulting in the even distribution of active components and the high density of active centers after calcination.

3. The forced circulation can drive solutions to flow into membrane pores in large quantities, improve the utilization of available loading sites in membrane pores, and remove the active components weakly bound with a ceramic membrane during the forced circulation, thereby enhancing the stability of the catalytic membrane.

Therefore, the Co@CM ceramic catalytic membrane prepared by the present disclosure exhibits high catalytic activity and high stability, can achieve the in-situ separation of a catalyst from a material, and is suitable for the large-scale industrial application.

### BRIEF DESCRIPTION OF THE DRAWINGS

FIG. 1 is a schematic diagram of a structure of a device adopted for the preparation and catalytic performance testing of the multi-channel Co@CM ceramic catalytic membrane in Example 1;

FIG. 2 is a schematic diagram of a membrane module and a forced circulation process;

FIG. 3 shows results of catalytic performance testing for multi-channel Co@CM ceramic catalytic membranes prepared under different solution introduction orders;

FIG. 4 shows an X-ray diffraction (XRD) pattern of the multi-channel Co@CM ceramic catalytic membrane in Example 1;

FIG. 5 shows energy-dispersive X-ray spectroscopy (EDX) results of the multi-channel Co@CM ceramic catalytic membrane in Example 1; and

FIG. 6 shows results of stability testing for the catalytic membrane in Example 1.

Reference numerals: 1—thermostatic water bath, 2—peristaltic pump, 3—peristaltic pump, 4—storage-tank jacket, 5—storage tank, 6—membrane-reactor jacket, 7—membrane module, 8—pressure gauge, 9—material feed pipe, 10—circulating water pipe, 11—normally closed exhaust valve, 12—sampling port, and 13—discharge port.

### DETAILED DESCRIPTION OF THE EMBODIMENTS

To make the objectives, features, and advantages of the present disclosure comprehensible, the present disclosure will be further described below with reference to the specific embodiments. It should be noted that the embodiments of the present disclosure and the features in the embodiments may be combined with each other in case of no conflict.

In the following description, many specific details are set forth in order to facilitate the full understanding of the present disclosure, but the present disclosure can also be implemented in other ways other than those described herein. Therefore, the present disclosure is not limited by the specific embodiments disclosed below.

### Example 1

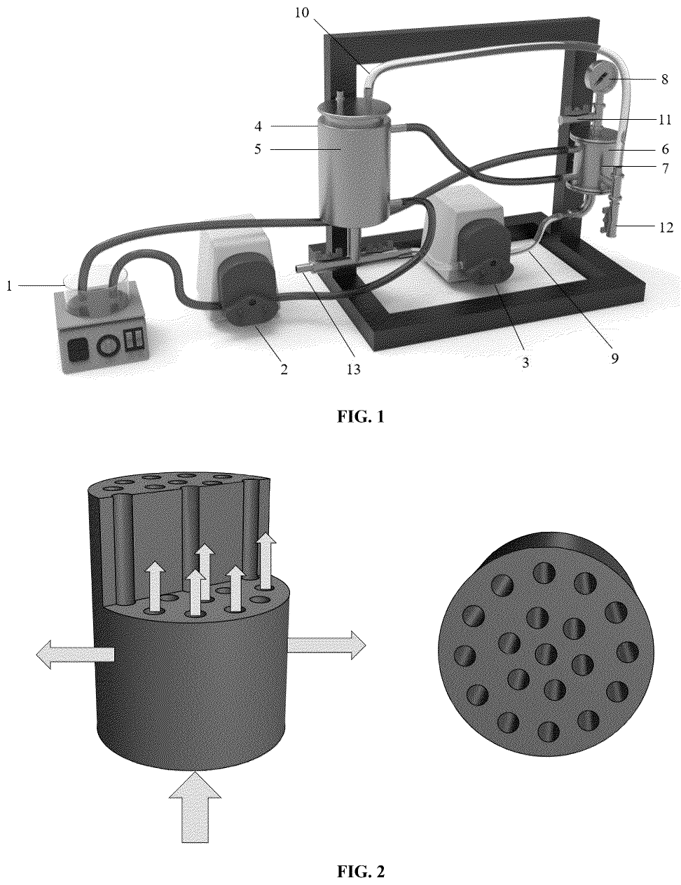

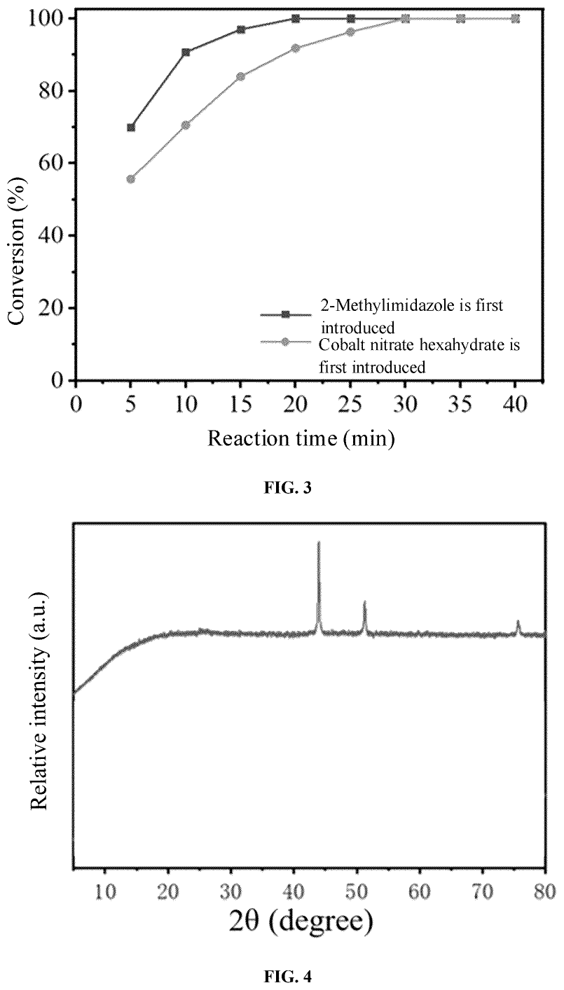

In this example and the following examples, the preparation of a catalytic membrane and the reaction for selective hydrogenation of p-nitrophenol to produce p-aminophenol were carried out with the device shown in FIG. 1 (The preparation of a catalytic membrane and the subsequent reaction may also be carried out with other devices. This example provides only one device capable of achieving both the preparation of a catalytic membrane and the subsequent reaction, and retains the possibility of other devices capable of implementing the technical solutions of the present disclosure. The designs of other devices are not defined here). FIG. 1 shows a typical structure of a flow-through membrane reactor, mainly including the following components: thermostatic water bath 1, storage tank 5, a membrane reactor, and a pipeline. Discharge port 13 configured to discharge a material is formed at a lower end of the storage tank 5. The entire device includes two circulations, namely a water circulation starting from the thermostatic water bath 1 and a material circulation starting from the storage tank. The membrane reactor and the storage tank 5 each are provided with a jacket externally. A lower end of the jacket is connected with an inlet piping of a thermostatic water pipe, and an upper end of the jacket is connected with an outlet piping of the thermostatic water pipe. Peristaltic pump 2 is provided between the thermostatic water bath 1 and the storage tank 5. After being pressurized by the peristaltic pump 2, thermostatic water flowing out of the thermostatic water bath 1 flows into storage-tank jacket 4 and membrane-reactor jacket 6 sequentially, then flows out from the membrane reactor, and then is returned through circulating water pipe 10 to the thermostatic water bath 1. A temperature of a reaction system is controlled by setting a temperature of the thermostatic water bath. Membrane module 7 (customized) is arranged in the membrane reactor, and a multi-channel ceramic membrane tube is arranged in the membrane module 7. A rubber ring is sleeved at two ends of the multi-channel ceramic membrane tube to fix the multi-channel ceramic membrane tube in the membrane module. Flanges at upper and lower ends are fixed on the membrane module by a quick-release clamp. A lower opening of the membrane module 7 is matched with material feed pipe 9 and serves as a liquid inlet. A side of the membrane module is matched with a material outlet pipe and serves as a discharge port. A top opening of the membrane module is matched with an evacuation pipeline and serves as a pressure relief port. Peristaltic pump 3 is provided on the material feed pipe 9, sampling port 12 is formed on the material outlet pipe, and pressure gauge 8 is provided on the evacuation pipeline. Normally closed exhaust valve 11 is provided at an outer side of the pressure gauge. When an upper end of the multi-channel ceramic membrane tube needs to be sealed, the normally closed exhaust valve 11 is closed.

A specific preparation process of a multi-channel Co@CM ceramic catalytic membrane was as follows:

### (1) Preparation of a ZIF-67@CM Membrane

200 mL of a solution of 0.48 M 2-methylimidazole in methanol (solution A) and 200 mL of a solution of 0.06 M cobalt nitrate hexahydrate in methanol (solution B) were prepared. Temperatures of the two solutions were controlled at 30° C. by a water bath until solid matters were completely dissolved. A multi-channel ceramic membrane tube (commercially-available, alumina, 19 channels, pore size: about 1,000 nm, diameter: 3 cm, and length: 8 cm) was filled in the membrane module. The solution of 2-methylimidazole in methanol was added to the storage tank 5 of the reactor. The temperature of the thermostatic water bath 1 was set to 30° C. The peristaltic pump 2 was turned on, and 5 min later, the peristaltic pump 3 was turned on. A flow rate was adjusted to 3.5 L/h. The solution A was forced to flow through membrane pores for 1 h. Then the solution A in the storage tank was poured out through the discharge port 13. The solution B was introduced instead and allowed to flow for 1 h. The introduction of the solution A and the solution B once was set as one cycle, and 2 cycles were conducted during the whole preparation process. 4 h later, the peristaltic pump 2 and the peristaltic pump 3 were turned off, the remaining solution in the reactor was discharged, and a methanol solution was added to the storage tank. The peristaltic pump 3 was turned on, and a flow rate was adjusted to 3.5 L/h. A resulting membrane tube was cleaned for 15 min, then taken out, and dried in a 60° C. oven for 18 h to produce a sample denoted as ZIF-67@CM-0.06.

### (2) Preparation of a Co@CM Catalyst

The ZIF-67@CM-0.06 was placed in a tube furnace, heated at a heating rate of 5° C./min to 550° C. from an initial temperature (room temperature) and kept at 550° C. for 5 h in an argon atmosphere to allow calcination, and then naturally cooled to room temperature to produce a sample denoted as Co@CM-550-0.06.

Membrane pores of the multi-channel ceramic catalytic membrane were rinsed to remove the active components unstably loaded. A specific operation method was as follows: A sufficient amount of a mixed solution of deionized water and ethanol (a volume ratio of the deionized water to the ethanol was 5:1), and added to the storage tank of the flow-through membrane reactor. The peristaltic pump 2 was turned on, and a flow rate was adjusted to 2.5 L/h. During this process, a solution flowing out of an outlet of the membrane module was not returned to the storage tank, but flowed out directly through the sampling port shown in FIG. 1. The entire process was carried out for 45 min. After the rinsing was completed, a multi-channel ceramic catalytic membrane with stable performance was produced. Because the rinsing solution had the same solvent as the reaction system, a catalytic membrane produced after the rinsing could be directly used for a catalytic reaction, or could be air-dried or oven-dried and stored.

In order to characterize and verify an effect of the prepared multi-channel ceramic catalytic membrane, a plurality of catalytic membranes were prepared under the same conditions in this example.

FIG. 4 shows an XRD pattern. The diffraction peak centered at 26.4° belongs to the (002) plane of graphitic carbon. The distinct derived peaks around 43.9°, 51.2°, and 75.6° belong to the (111), (200), and (220) crystal planes of metallic Co, respectively, indicating that Co in ZIF-67 is reduced into zero-valent Co during pyrolysis and serves as a catalytic active center.

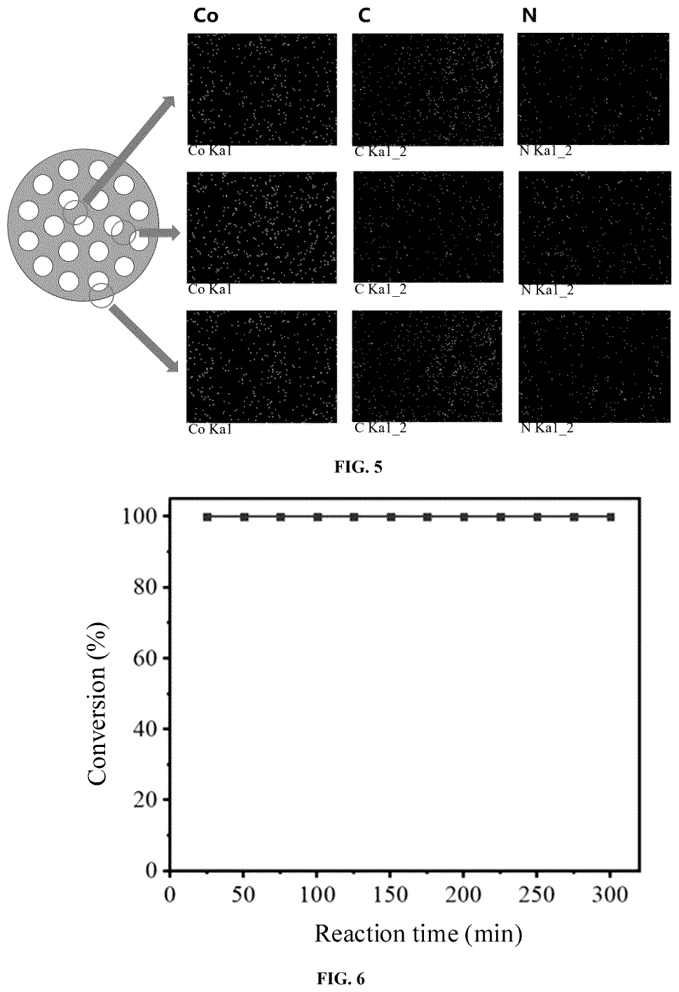

FIG. 5 shows the distribution of Co, C, and N elements in the catalytic membrane prepared in Example 1. It can be seen from this figure that ZIF-67 is pyrolyzed into metal Co after calcination, and the Co, C, and N elements exist in each layer of the multi-channel ceramic membrane and are evenly distributed. As a result, the multi-channel ceramic membrane can provide abundant loading sites for Co, which is the basis for the catalytic membrane to efficiently catalyze the hydrogenation of p-nitrophenol to produce p-aminophenol and enable a simple in-situ reduction process.

The catalytic membrane Co@CM-550-0.06 prepared in this example was used in an experiment for hydrogenation of p-nitrophenol to produce p-aminophenol, and the catalytic performance of the catalytic membrane was tested with the flow-through membrane reactor. The thermostatic water bath was turned on, and a temperature of the thermostatic water bath was set to 50° C. Once the temperature in the thermostatic water bath reached the set value, the peristaltic pump 2 was turned on to make thermostatic water flow into the storage-tank jacket and the membrane-reactor jacket sequentially. Then a mixed reaction raw material was prepared as follows: 1 g of p-nitrophenol was added to 240 mL of a mixed solvent of ethanol and deionized water (a volume ratio of the ethanol to the deionized water was 1:5), and stirring was conducted manually until p-nitrophenol was completely dissolved to produce a reaction solution. 0.4 mL of the reaction solution was taken as an initial sample. Then 3.915 g of NaBH₄ was then added, and stirring was conducted manually for 5 min to 10 min until the solid was dissolved to produce the mixed reaction raw material. The mixed reaction raw material was added to the storage tank of the flow-through membrane reactor. The peristaltic pump 3 was turned on, and a flow rate was adjusted to 3.5 L/h. When a reaction solution flowed to the outlet of the membrane module, the timing was started. After flowing out of the membrane module, the reaction solution circularly flowed into the storage tank. 0.4 mL of the reaction solution was collected every 5 min. After a reaction was completed, a liquid in the storage tank was discharged, and deionized water was introduced instead for a forced circulation to wash membrane pores for 10 min to 15 min. A resulting membrane tube was taken out, air-dried or oven-dried, and stored. Alternatively, a fresh raw material was added to continue the reaction. A composition of a product was detected by HPLC (Agilent 1200). The conversion and selectivity of the reaction were then calculated according to a standard curve.

Table 1 shows the turnover frequency (TOF) values of the Co@CM catalytic membrane and non-precious metal catalysts reported in the literature in recent years. It can be seen from Table 1 that the Co@CM ceramic catalytic membrane obtained in this example still has a high TOF value (1.69×10⁻² s⁻¹) with a small consumption of NaBH₄, indicating that the Co@CM catalytic membrane has excellent catalytic performance and a promising application prospect.

TABLE 1
TOF values of the Co@CM catalytic membrane and catalysts reported in the literature

| | | Reaction | Conversion | |
|---|---|---|---|---|
| Catalyst | Reaction conditions | time (s) | (%) | TOF (s⁻¹) |
| Co-NCC | p-Nitrophenol concentration: 0.17 | 115 | 100 | 5.31 × 10⁻⁴ |
| | mM; | | | |
| | Molar ratio of sodium borohydride | | | |
| | to p-nitrophenol: 1,190; | | | |
| | Room temperature | | | |
| C@Co | p-Nitrophenol concentration: 0.02 | 120 | 99.34 | 4.73 × 10⁻⁵ |
| | mM; | | | |
| | Molar ratio of sodium borohydride | | | |
| | to p-nitrophenol: 500; | | | |
| BNPC-1000 | p-Nitrophenol concentration: 0.1 | 1200 | 95.2 | 1.412 × 10⁻⁴ |
| | mM; | | | |
| | Molar ratio of sodium borohydride | | | |
| | to p-nitrophenol: 2,500; | | | |
| Co@CM-550-0.06 | p-Nitrophenol concentration: 7.19 | 1200 | 100 | 1.69 × 10⁻² |
| (Example 1) | mM; | | | |
| | Molar ratio of sodium borohydride | | | |
| | to p-nitrophenol: 14.4; 50° C. | | | |

Note:
TOF is calculated as an amount of p-nitrophenol reduced per mole of an active component per hour.

### Example 2

### (1) Preparation of a ZIF-67@CM Membrane

200 mL of a solution of 0.32 M 2-methylimidazole in methanol and 200 mL of a solution of 0.04 M cobalt nitrate hexahydrate in methanol were prepared. The two solutions each were placed in a water bath with a temperature controlled at 30° C. until solid matters were completely dissolved. A multi-channel ceramic membrane tube (commercially-available, zirconia, 7 channels, pore size: about 200 nm, diameter: 3 cm, and length: 8 cm) was filled in the membrane module. The solution of 2-methylimidazole in methanol was added to the storage tank of the reactor. A temperature was controlled at 25° C. through the thermostatic water bath. The peristaltic pumps were turned on, and a flow rate was adjusted to 2.5 L/h. The solution A was forced to flow through membrane pores for 1 h. Then the solution A in the reactor was replaced with the solution B which was allowed to flow for 1 h. The introduction of the solution A and the solution B once was set as one cycle, and 2 cycles were conducted during the whole preparation process. 4 h later, the remaining solution in the reactor was discharged, and a methanol solution was added to the storage tank. The peristaltic pump 3 was turned on, and a flow rate was adjusted to 2.5 L/h. A resulting membrane tube was cleaned for 10 min, then taken out, and dried in a 70° C. oven for 12 h to produce a sample denoted as ZIF-67@CM-0.04.

### (2) Preparation of a Co@CM Catalyst

The ZIF-67@CM-0.04 was placed in a tube furnace, and heated at a heating rate of 2° C./min to a target temperature of 450° C. and kept at the target temperature for 4 h in an argon atmosphere to produce a sample denoted as Co@CM-450-0.04.

The catalytic membrane Co@CM was used in an experiment for hydrogenation of p-nitrophenol to produce p-aminophenol. A corresponding operation method was the same as the operation method in Example 1. When a reaction was conducted for 20 min, the conversion reached 84.7% and the selectivity was 100%.

### Example 3

### (1) Preparation of a ZIF-67@CM Membrane

200 mL of a solution of 0.64 M 2-methylimidazole in methanol (solution A) and 200 mL of a solution of 0.08 M cobalt nitrate hexahydrate in methanol (solution B) were prepared in two 500 mL beakers, respectively, and then placed in a water bath with a temperature controlled at 30° C. until solid matters were completely dissolved. A multi-channel ceramic membrane tube (commercially-available, alumina, 37 channels, pore size: about 5,000 nm, diameter: 3 cm, and length: 8 cm) was filled in the membrane module. The solution of 2-methylimidazole in methanol was added to the storage tank of the reactor. A temperature was controlled at 45° C. through the thermostatic water bath. The peristaltic pumps were turned on, and a flow rate was adjusted to 4.5 L/h. The solution A was forced to flow through membrane pores for 1 h. Then the solution A in the reactor was replaced with the solution B which was allowed to flow for 1 h. The introduction of the solution A and the solution B once was set as one cycle, and 2 cycles were conducted during the whole preparation process. 4 h later, the remaining solution in the reactor was discharged, and a methanol solution was added. A resulting membrane tube was cleaned for 20 min, then taken out, and dried in a 50° C. oven for 24 h to produce a sample denoted as ZIF-67@CM-0.10.

### (2) Preparation of a Co@CM Catalyst

The ZIF-67@CM-0.10 was placed in a tube furnace, and heated at a heating rate of 10° C./min to a target temperature of 770° C. from an initial temperature (room temperature) and kept at the target temperature for 6 h in an argon atmosphere to produce a sample denoted as Co@CM-770-0.10.

The catalytic membrane Co@CM was used in an experiment for hydrogenation of p-nitrophenol to produce p-aminophenol. A corresponding operation method was the same as the operation method in Example 1. When a reaction was conducted for 20 min, the conversion reached 92.3% and the selectivity was 100%.

### Example 4

### (1) Preparation of a ZIF-67@CM Membrane

200 mL of a solution of 0.64 M 2-methylimidazole in methanol (solution A) and 200 mL of a solution of 0.08 M cobalt nitrate hexahydrate in methanol (solution B) were prepared in two 500 mL beakers, respectively, and then placed in a water bath with a temperature controlled at 30° C. until solid matters were completely dissolved. A multi-channel ceramic membrane tube (commercially-available, alumina, 61 channels, pore size: about 1,500 nm, diameter: 3 cm, and length: 8 cm) was filled in the membrane module. The solution of 2-methylimidazole in methanol was added to the storage tank of the reactor. A temperature was controlled at 30° C. through the thermostatic water bath. The peristaltic pumps were turned on, and a flow rate was adjusted to 5.5 L/h. The solution A was forced to flow through membrane pores for 1 h. Then the solution A in the reactor was replaced with the solution B which was allowed to flow for 1 h. The introduction of the solution A and the solution B once was set as one cycle, and 2 cycles were conducted during the whole preparation process. 4 h later, the remaining solution in the reactor was discharged, and a methanol solution was added. A resulting membrane tube was cleaned for 15 min, then taken out, and dried in a 60° C. oven for 12 h to produce a sample denoted as ZIF-67@CM-0.08.

### (2) Preparation of a Co@CM Catalyst

The ZIF-67@CM-0.08 was placed in a tube furnace, and heated at a heating rate of 5° C./min to a target temperature of 650° C. from an initial temperature (room temperature) and kept at the target temperature for 5 h in an argon atmosphere to produce a sample denoted as Co@CM-650-0.08.

The catalytic membrane Co@CM was used in an experiment for hydrogenation of p-nitrophenol to produce p-aminophenol. A corresponding operation method was the same as the operation method in Example 1. When a reaction was conducted for 20 min, the conversion reached 85% and the selectivity was 100%.

### Example 5

In this example, the Co@CM-550-0.06 prepared in Example 1 was tested for catalytic stability. Before the stability testing, with the device shown in FIG. 1, membrane pores of the multi-channel ceramic catalytic membrane were rinsed to remove the active components unstably loaded. A specific operation method was as follows: A sufficient amount of a mixed solution of deionized water and ethanol (a volume ratio of the deionized water to the ethanol was 5:1), and added to the storage tank of the flow-through membrane reactor. The peristaltic pump 2 was turned on, and a flow rate was adjusted to 2.5 L/h. During this process, a solution flowing out of an outlet of the membrane module was not returned to the storage tank, but flowed out directly through the sampling port shown in FIG. 1. The entire process was carried out for 45 min. After the process was completed, a resulting multi-channel ceramic catalytic membrane could be directly used for the stability testing in this example (namely a continuous catalytic process), or could be air-dried or oven-dried and stored.

A specific method of the stability testing was as follows: A sufficient amount of a 0.5 g/L p-nitrophenol solution was prepared, sodium borohydride was added after a solid matter was completely dissolved (a molar ratio of the p-nitrophenol to the sodium borohydride was 1:14.4), and stirring was conducted until the solid was dissolved. A temperature of the thermostatic water bath was set to 50° C. Once the temperature reached the set value, the peristaltic pump 2 was turned on to make thermostatic water flow into the storage-tank jacket and the membrane-reactor jacket sequentially. A reaction raw material was added to the storage tank of the flow-through membrane reactor, the peristaltic pump 3 was turned on, and a flow rate was adjusted to 3.5 L/h. During this process, a solution flowing out from the outlet of the membrane module was not returned to the storage tank, but flowed out directly through the sampling port shown in FIG. 1. A receiving tank could be additionally provided to hold a reaction product. A sample was collected every 25 min at the sampling port. During this process, when a solution in the storage tank was about to be exhausted, the solution prepared above was filled in the storage tank, and a reaction was continued for 5 h. As shown in FIG. 6, a conversion of the catalytic membrane for p-nitrophenol was maintained at 100% within 5 h, and there was no obvious inactivation.

### Comparative Example 1

### (1) Preparation of a ZIF-67@CM Membrane

200 mL of a solution of 0.48 M 2-methylimidazole in methanol (solution A) and 200 mL of a solution of 0.06 M cobalt nitrate hexahydrate in methanol (solution B) were prepared in two 500 mL beakers, respectively, and then placed in a water bath with a temperature controlled at 30° C. until solid matters were completely dissolved. A multi-channel ceramic membrane tube (commercially-available, alumina, 19 channels, pore size: about 1,000 nm, diameter: 3 cm, and length: 8 cm) was filled in the membrane module. The solution of 2-methylimidazole in methanol was added to the storage tank of the reactor. A temperature was controlled at 30° C. through the thermostatic water bath. The peristaltic pumps were turned on, and a flow rate was adjusted to 3.5 L/h. The solution A was forced to flow through membrane pores for 1 h. Then the solution A in the reactor was replaced with the solution B which was allowed to flow for 1 h. The introduction of the solution A and the solution B once was set as one cycle, and 2 cycles were conducted during the whole preparation process. 4 h later, the remaining solution in the reactor was discharged, and a methanol solution was added. A resulting membrane tube was cleaned for 10 min, then taken out, and dried in a 70° C. oven for 24 h to produce a sample denoted as ZIF-67@CM-0.06.

### (2) Preparation of a Co@CM Catalyst

The ZIF-67@CM-0.06 was placed in a tube furnace, and heated at a heating rate of 5° C./min to a target temperature of 350° C. from an initial temperature (room temperature) and kept at the target temperature for 5 h in an argon atmosphere to produce a sample denoted as Co@CM-350-0.06.

The catalytic membrane Co@CM was used in an experiment for hydrogenation of p-nitrophenol to produce p-aminophenol. A corresponding operation method was the same as the operation method in Example 1. When a reaction was conducted for 20 min, the conversion was 53.1% and the selectivity was 100%.

### Comparative Example 2

### (1) Preparation of a ZIF-67@CM Membrane

200 mL of a solution of 0.16 M 2-methylimidazole in methanol (solution A) and 200 mL of a solution of 0.02 M cobalt nitrate hexahydrate in methanol (solution B) were prepared in two 500 mL beakers, respectively, and then placed in a water bath with a temperature controlled at 30° C. until solid matters were completely dissolved. A multi-channel ceramic membrane tube (commercially-available, alumina, 19 channels, pore size: about 1,000 nm, diameter: 3 cm, and length: 8 cm) was filled in the membrane module. The solution of 2-methylimidazole in methanol was added to the storage tank of the reactor. A temperature was controlled at 30° C. through the thermostatic water bath. The peristaltic pumps were turned on, and a flow rate was adjusted to 3.5 L/h. The solution A was forced to flow through membrane pores for 1 h. Then the solution A in the reactor was replaced with the solution B which was allowed to flow for 1 h. The introduction of the solution A and the solution B once was set as one cycle, and 2 cycles were conducted during the whole preparation process. 4 h later, the remaining solution in the reactor was discharged, and a methanol solution was added. A resulting membrane tube was cleaned for 10 min, then taken out, and dried in a 70° C. oven for 24 h to produce a sample denoted as ZIF-67@CM-0.02.

### (2) Preparation of a Co@CM Catalyst

The ZIF-67@CM-0.02 was placed in a tube furnace, and heated at a heating rate of 5° C./min to a target temperature of 550° C. from an initial temperature (room temperature) and kept at the target temperature for 5 h in an argon atmosphere to produce a sample denoted as Co@CM-550-0.02.

The catalytic membrane Co@CM was used in an experiment for hydrogenation of p-nitrophenol to produce p-aminophenol. A corresponding operation method was the same as the operation method in Example 1. When a reaction was conducted for 20 min, the conversion was 42.4% and the selectivity was 100%.

The above are only preferred examples of the present disclosure, and are not intended to limit the present disclosure in other forms. Any person skilled in the art may change or modify the technical content disclosed above into an equivalent example to be applied in other fields.

Any simple amendment or equivalent change and modification of the above example made according to the technical essence of the present disclosure without departing from the content of the technical solutions of the present disclosure shall fall within the protection scope of the technical solutions of the present disclosure.

What is claimed is:

1. A preparation method of a multi-channel Co@CM ceramic catalytic membrane, comprising the following steps:
step 1, dissolving 2-methylimidazole in methanol to produce a first solution;
step 2, dissolving cobalt nitrate hexahydrate in methanol to produce a second solution;
step 3, closing an end of a multi-channel ceramic membrane tube; filling channels of the multi-channel ceramic membrane tube with the first solution, and applying a pressure to force a circulation flow of the first solution from insides of the channels of the multi-channel ceramic membrane tube to an outside of the multi-channel ceramic membrane tube for 1 h or more; replacing the first solution with the second solution, and forcing a circulation flow of the second solution from the insides of the channels of the multi-channel ceramic membrane tube to the outside of the multi-channel ceramic membrane tube for 1 h or more; and iterating such that at least two cycles of alternate forced circulation flows of the first solution and the second solution are achieved;
step 4, forcing methanol to flow from the insides of the channels of the multi-channel ceramic membrane tube to the outside of the multi-channel ceramic membrane tube for washing, and oven-drying to produce a multi-channel ZIF-67@CM ceramic membrane;
step 5, calcining the multi-channel ZIF-67@CM ceramic membrane; and
step 6, rinsing membrane pores through a forced circulation of an ethanol aqueous solution to produce the multi-channel Co@CM ceramic catalytic membrane.

2. The preparation method of the multi-channel Co@CM ceramic catalytic membrane according to claim 1, wherein a concentration of the 2-methylimidazole in the first solution is 0.32 mol/L to 0.64 mol/L; and a concentration of the cobalt nitrate hexahydrate in the second solution is 0.04 mol/L to 0.08 mol/L.

3. The preparation method of the multi-channel Co@CM ceramic catalytic membrane according to claim 1, wherein in the step 3, a material for the multi-channel ceramic membrane tube is alumina or zirconia, and the multi-channel ceramic membrane tube comprises 7 to 61 channels and has a pore size of 200 nm to 5,000 nm; temperatures of the first solution, the second solution, and the multi-channel ceramic membrane tube are kept at 25° C. to 45° C.; and a total time of the at least two cycles of alternate forced circulation flows of the first solution and the second solution is 4 h to 6 h, and a flow rate for the alternate forced circulation flows of the first solution and the second solution is 2.5 L/h to 5.5 L/h.

4. The preparation method of the multi-channel Co@CM ceramic catalytic membrane according to claim 1, wherein in the step 4, the washing is conducted for 10 min to 20 min, and the oven-drying is conducted at 50° C. to 70° C. for 12 h to 24 h.

5. The preparation method of the multi-channel Co@CM ceramic catalytic membrane according to claim 1, wherein in the step 5, a pyrolysis temperature is 450° C. to 770° C., a calcination atmosphere is argon, a heating rate is 2° C./min to 10° C./min, and the pyrolysis temperature is held for 4 h to 6 h.

6. The preparation method of the multi-channel Co@CM ceramic catalytic membrane according to claim 1, wherein in the step 6, a flow rate of the forced circulation for the rinsing is 2.5 L/h; the rinsing is conducted for 45 min with the ethanol aqueous solution; and in the ethanol aqueous solution, a volume ratio of deionized water to ethanol is 5:1.

7. A process for selective hydrogenation of p-nitrophenol to produce p-aminophenol, comprising: immersing the multi-channel Co@CM ceramic catalytic membrane prepared by the preparation method according to claim 1 as a catalyst in a p-nitrophenol solution to allow an intermittent or continuous reaction.

8. The process for selective hydrogenation of p-nitrophenol to produce p-aminophenol according to claim 7, wherein a recovered multi-channel Co@CM ceramic catalytic membrane is used to catalyze the p-nitrophenol solution to allow the intermittent or continuous reaction; and a recovery method is as follows: washing the membrane pores for 10 min to 15 min through a forced circulation of deionized water, and air-drying or oven-drying a resulting ceramic membrane.

9. The process for selective hydrogenation of p-nitrophenol to produce p-aminophenol according to claim 7, wherein in the preparation method, a concentration of the 2-methylimidazole in the first solution is 0.32 mol/L to 0.64 mol/L; and a concentration of the cobalt nitrate hexahydrate in the second solution is 0.04 mol/L to 0.08 mol/L.

10. The process for selective hydrogenation of p-nitrophenol to produce p-aminophenol according to claim 7, wherein in the step 3 of the preparation method, a material for the multi-channel ceramic membrane tube is alumina or zirconia, and the multi-channel ceramic membrane tube comprises 7 to 61 channels and has a pore size of 200 nm to 5,000 nm; temperatures of the first solution, the second solution, and the multi-channel ceramic membrane tube are kept at 25° C. to 45° C.; and a total time of the at least two cycles of alternate forced circulation flows of the first solution and the second solution is 4 h to 6 h, and a flow rate for the alternate forced circulation flows of the first solution and the second solution is 2.5 L/h to 5.5 L/h.

11. The process for selective hydrogenation of p-nitrophenol to produce p-aminophenol according to claim 7, wherein in the step 4 of the preparation method, the washing is conducted for 10 min to 20 min, and the oven-drying is conducted at 50° C. to 70° C. for 12 h to 24 h.

12. The process for selective hydrogenation of p-nitrophenol to produce p-aminophenol according to claim 7, wherein in the step 5 of the preparation method, a pyrolysis temperature is 450° C. to 770° C., a calcination atmosphere is argon, a heating rate is 2° C./min to 10° C./min, and the pyrolysis temperature is held for 4 h to 6 h.

13. The process for selective hydrogenation of p-nitrophenol to produce p-aminophenol according to claim 7, wherein in the step 6 of the preparation method, a flow rate of the forced circulation for the rinsing is 2.5 L/h; the rinsing is conducted for 45 min with the ethanol aqueous solution; and in the ethanol aqueous solution, a volume ratio of deionized water to ethanol is 5:1.

14. The process for selective hydrogenation of p-nitrophenol to produce p-aminophenol according to claim 9, wherein a recovered multi-channel Co@CM ceramic catalytic membrane is used to catalyze the p-nitrophenol solution to allow the intermittent or continuous reaction; and a recovery method is as follows: washing the membrane pores for 10 min to 15 min through a forced circulation of deionized water, and air-drying or oven-drying a resulting ceramic membrane.

15. The process for selective hydrogenation of p-nitrophenol to produce p-aminophenol according to claim 10, wherein a recovered multi-channel Co@CM ceramic catalytic membrane is used to catalyze the p-nitrophenol solution to allow the intermittent or continuous reaction; and a recovery method is as follows: washing the membrane pores for 10 min to 15 min through a forced circulation of deionized water, and air-drying or oven-drying a resulting ceramic membrane.

16. The process for selective hydrogenation of p-nitrophenol to produce p-aminophenol according to claim 11, wherein a recovered multi-channel Co@CM ceramic catalytic membrane is used to catalyze the p-nitrophenol solution to allow the intermittent or continuous reaction; and a recovery method is as follows: washing the membrane pores for 10 min to 15 min through a forced circulation of deionized water, and air-drying or oven-drying a resulting ceramic membrane.

17. The process for selective hydrogenation of p-nitrophenol to produce p-aminophenol according to claim 12, wherein a recovered multi-channel Co@CM ceramic catalytic membrane is used to catalyze the p-nitrophenol solution to allow the intermittent or continuous reaction; and a recovery method is as follows: washing the membrane pores for 10 min to 15 min through a forced circulation of deionized water, and air-drying or oven-drying a resulting ceramic membrane.

18. The process for selective hydrogenation of p-nitrophenol to produce p-aminophenol according to claim 13, wherein a recovered multi-channel Co@CM ceramic catalytic membrane is used to catalyze the p-nitrophenol solution to allow the intermittent or continuous reaction; and a recovery method is as follows: washing the membrane pores for 10 min to 15 min through a forced circulation of deionized water, and air-drying or oven-drying a resulting ceramic membrane.
